(19) United States
(12) Patent Application Publication
(10) Pub. No.: US 2017/0049967 A1
(43) Pub. Date: Feb. 23, 2017

(54) NEEDLE FREE INJECTION DEVICE

(71) Applicant: SRI International, Menlo Park, CA (US)

(72) Inventors: David Fouche, Menlo Park, CA (US); Michael Vestel, Menlo Park, CA (US); John P. Marlow, Menlo Park, CA (US); Mark A. Petrie, Menlo Park, CA (US); Robert A. Brown, Menlo Park, CA (US); Hilary Lackritz, Menlo Park, CA (US); Richard A. Hill, Menlo Park, CA (US); Donald English, Menlo Park, CA (US); Mark Nansen, Menlo Park, CA (US); Mark Hansen, Menlo Park, CA (US); Martin Martoza, Menlo Park, CA (US)

(21) Appl. No.: 15/343,853

(22) Filed: Nov. 4, 2016

Related U.S. Application Data
(63) Continuation of application No. PCT/US2015/029794, filed on May 7, 2015.
(60) Provisional application No. 61/990,003, filed on May 7, 2014.

(51) Int. Cl. A61M 5/30; A61M 5/00

(52) U.S. Cl. CPC A61M 5/3007; A61M 5/007; A61M 2205/8231

(57) ABSTRACT

Devices, systems and methods for needle free injection include a multi-use needle-free injection device comprising reversibly connected proximate and distal sections, wherein the proximate section comprises a power supply, actuator, circuitry and a housing, and the distal section comprises a disposable nozzle tip prefilled with an injectate, a chemical energetic device, and a dual seal piston barrier between the energetic device and the injectate, wherein the proximate and distal sections and their components are operably linked and configured for single-hand injecting of the injectate.

### CROSS-REFERENCE TO RELATED APPLICATIONS

The present application is a continuation of, claims the benefit of, and incorporates herein by reference, in its entirety, International Application Number PCT/US2015/029794 (International Publication Number WO 2015/171964), filed on May 7, 2015, and entitled “NEEDLE FREE INJECTION DEVICE”, which is based on, claims the benefit of, and incorporates herein by reference, in its entirety, U.S. Provisional Application Ser. No. 61/990,003 filed on May 7, 2014, and entitled “NEEDLE FREE INJECTION DEVICE.”

### CROSS-REFERENCE TO RELATED APPLICATIONS

This invention was made with government support under HR0011-12-C-0042 awarded by Defense Advanced Research Projects Agency. The government has certain rights in the invention.

### BACKGROUND

Many prior needle free injectors are heavy and difficult to use because of large springs or compressed gas chambers that are necessary to generate pressure required for operation. In addition, prior devices are not configured to implement prefilled vials, and require complicated steps to use. Furthermore, prior devices are insufficiently robust for repetitive field use, and/or incapable of diverse injection modalities, such as intramuscular, subcutaneous and intradermal injection.

Therefore, there is a need for improved technologies directed to needle free injection.

### SUMMARY

A Variable, Injector, Safe, Tailorable, Affordable (VISTA) jet device is herein introduced which is a safe, easy-to-use needle free hand-held injection delivery system configured with prefilled disposable tips. The device need not use needles, and uses chemically produced pressure to deliver an injectate, such as a drug or medicine, in the form of a high velocity microjet that penetrates the skin of a subject. In some aspects, the bottom end of the device comprises a disposable nozzle assembly, typically plastic, that can be prefilled with injectate. A small energetic device or energetic material may also be included or sealed into the nozzle assembly, and activated using an electric circuit to deliver the injectate through a nozzle in the nozzle assembly. The nozzle assembly also includes a dual seal piston, as a barrier between the energetic device and the injectate.

The top end of the device comprises circuitry for controlling the delivery of the injectate. Specifically, the top end includes a power supply, such as batteries, and other circuitry including capacitors, and switches. When the switches are activated, charged capacitors discharge current that activates the energetic device. Once activated, the resultant pressure gradient moves the dual seal piston to pressurize the injectate, which exits out the nozzle in the nozzle assembly and can then penetrate the skin.

The amount of energetic material, expansion volume, and nozzle configuration can be tailored to specific drugs and depths of penetration in tissue. The disposable nozzle assembly may be contained in a safety sleeve, typically a metal like stainless steel or aluminum or durable plastic, which may lock into the top of the device, using a connector, for example. Such connector may be a bayonet style connector and/or comprise pins protruding from the sleeve that align and reversibly lock into the top end of the device, such as by rotating a lock ring with angled channels. The electrical leads for the energetic device may connect to the top end via pin connectors. The VISTA jet is designed to be reusable for multi-use injections with the exception of the disposable tips which are designed for a one time use.

The invention specifically provides all combinations of the recited embodiments, as if each had been laboriously individually set forth.

### BRIEF DESCRIPTION OF THE DRAWINGS

FIG. 1 shows a side view of the top end of a needle free injection device, in accordance with the present disclosure.

FIG. 2 shows a side view of the bottom end of a needle free injection device, in accordance with the present disclosure.

FIG. 3 shows side views of different embodiments for the disposable tip and sleeve used in a needle free injection device, in accordance with the present disclosure.

### DETAILED DESCRIPTION

To address the limitations of previous technologies, the present disclosure introduces a hand-held injection delivery system that is simple, easy to use, safe and tailorable. The present injection delivery system, herein referred to as a Variable, Injector, Safe, Tailorable, Affordable (VISTA) jet device, is needle free injection device that is configured for single-hand operation and has a broad range of application. For instance anyone using syringes or needle free injection devices, such as field medics, doctors nurses, diabetic patients, veterinarians, emergency personnel, EMTs, firefighters, police, ER personnel, nursing home staff, and others, may benefit from the present VISTA jet device.

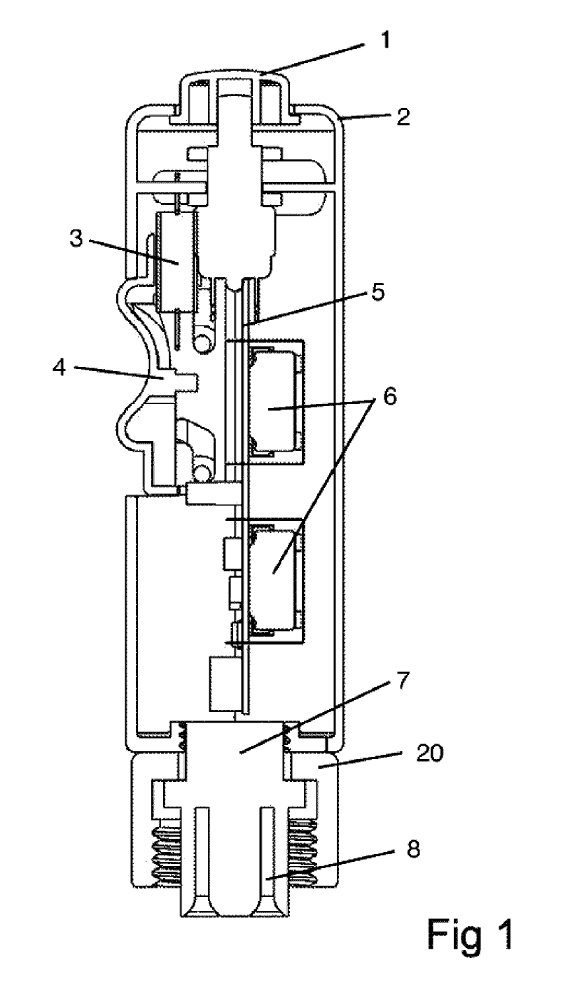

The VISTA jet device generally includes a reusable top end, or proximate section, and a disposable bottom end, or distal section. As shown in FIG. 1, the top end may include a push button (1), a housing (2), a safety spring mechanism (3), a side button (4), a circuit board (5), one or more power supplies (6), an integration part (7), one or more receptacles for initiator prongs (8), a slip ring (20), and other circuitry and components.

As shown, inside the housing (2) is the circuit board (5) that includes an injection switch or actuator controlled by the push button (1) on top, and a charging switch controlled by the side button (4) on the side. The housing (2) was 3D printed for the initial prototype, yet could be produced from aluminum to provide a more robust fabrication. The housing (2) accepts the threads from the integration part (7) on the bottom.

The side button (4) is configured to provide safety. It is designed with slots that ride on pins in the housing (2) and can activate a single pole double throw switch on the circuit board (5). The default state is spring loaded in the up and out position, and the switch shorts the connectors that accept the initiator for safety. When the side button (4) is engaged by pushing it down and in, it activates the charging switch on the circuit board (5) to charge the capacitors in the circuit, thereby arming the device to make it ready to fire. When the push button (1) is subsequently pressed, the device can fire. However, if the push button (1) is pressed when the side button (4) is not engaged, the device will not fire.

The circuit board (5) may be custom designed and fabricated to allow the capacitors, charging circuit, battery holders, batteries (LR44) (6), and shorting/charging switches to be mounted thereto.

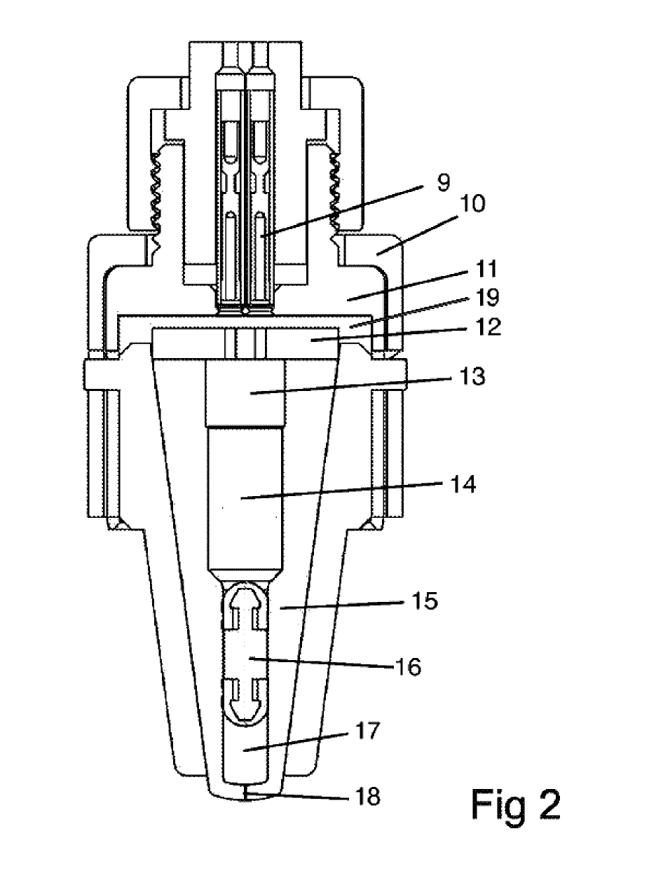

The integration part (7) houses the female connectors for the initiator leads (9), as shown in FIG. 2, and mating alignment holes for the plug pins to guide the connectors into the plug counter bores. The integration part (7) screws into the top of the top end body and has a shoulder to hold the slip ring (20) on. The first prototype was 3D printed out of UV curable material, however aluminum may be used to provide a more robust fabrication.

The slip ring (20) screws onto the top of the plug (11), holding the integration part (7) against the top end (12) of the plug (11) (as seen in FIG. 2), and holding the top end to the bottom end as well. Since the slip ring (20) is designed to be screwed onto the plug (11) and not removed, thread sealer could be used during assembly. The first prototype was 3D printed out of UV curable material, however aluminum may be used to provide a more robust fabrication.

As shown in FIG. 2, the bottom end may include a disposable nozzle assembly, a lock ring (10) and plug (11). The nozzle assembly includes a top end (12), an igniter (13), an expansion chamber (14), a nozzle body (15), a plunger (16), a nozzle tip prefilled with an injectant (17), a nozzle (18), and a cap (19). The nozzle assembly may be encased or inserted into a sleeve.

The igniter (13) or initiator may include any energetic source or device having an energetic material to power the VISTA jet device. For example, the igniter (13) may be a chemical igniter such as part #C5K14276_NC (Special Devices Inc) that includes 35 mg of the chemicals Zirconium/Potassium Perchlorate (ZPP). The ZPP ignition reaction is fully contained in the nozzle assembly to prevent any gasses, or other products produced from the chemical reaction, to escape. The ZPP may be bound with Viton on a nichrome wire/heater in the igniter with two leads protruding through the top end (12). The ZPP needs at least a temperature of 350 C to ignite. The nichrome wire/heater can provide this temperature reliably with 1.2 amps for 2 ms or 1.75 amps for 0.5 ms. Specifically, ZPP igniters are designed to be inert from static electricity, and are safe if 0.4 amps or less flows through the terminals for any amount of time.

The nozzle body (15) may be a plastic or thermoplastic that can sustain a high temperature, have high strength, and can be injection molded or machined, such as Ultem plastic (polyetherimide). As such, the nozzle assembly can fully contain the products and pressure during ignition. The inner diameter (ID) of the nozzle assembly toward the top end (12) could be configured to accept the igniter (13). Below the igniter (13) space is an empty volume, or expansion chamber (14), which allows the energetic material to expand during the ignition reaction. The expansion chamber (14) can have a slightly smaller inner diameter compared to the igniter (13). The smaller the volume of the expansion chamber (14), the higher the pressure the initiator will create. The volume can be modified to tailor the pressure, and the two initial engineered volumes were designed to create about 3000 psi for the low pressure design and 6000 psi for the high pressure design. Below the larger volume expansion chamber (14) is a smaller bore (0.180″) that holds the plunger (16) and the drug or injectant (17) on the other side of the plunger (16). At the end of the 0.180″ bore there is a nozzle (18) through which the drug can exit when pressurized. The nozzle (18) can include one or more openings or orifices.

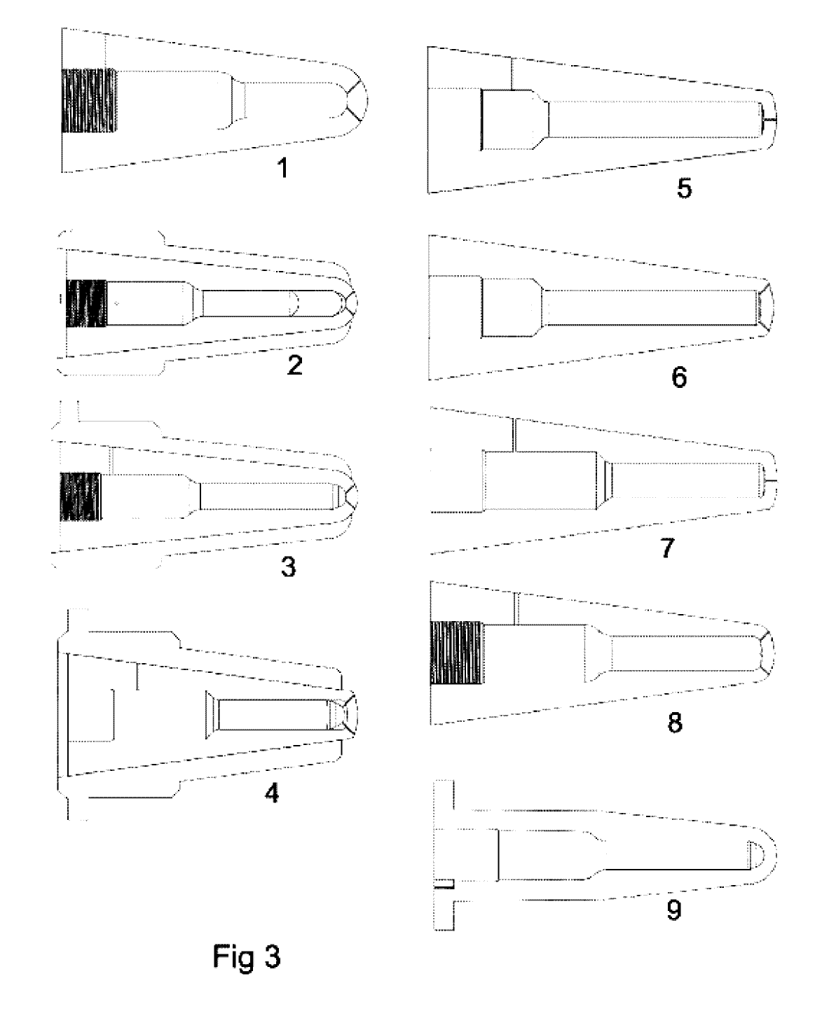

Nozzles and nozzle assembly architectures (as shown in FIG. 3) can be engineered differently to modulate to different injection depths and spread. For instance, a single, large diameter opening extending straight out of the nozzle (18) will inject the injectant (17) at the largest depth, while a nozzle (18) with one or more smaller opening that extends radially around the tip of the nozzle (18) will inject shallower. The baseline designs that were tested had several nozzle configurations. For instance, nozzles (18) having single openings that extended straight out of the tip of the nozzle (18) included diameters of 6, 7, 8, 10, 12, and 16 mil. Also, in another configuration, four radial openings of 6 mil diameter that extended at a 45 degree angle were tested. Openings with larger diameters would increase the area of the injectant. The outside of the nozzle (18) can be tapered at an angle for ease of loading and unloading in the sleeve. The nozzle assembly can be prefilled with a single, predetermined dose of a drug or the injectant (17). The nozzle (18) may be sterilized and filled in a sterile environment. A sterile cap can cover the end of the nozzle (18) keeping it sterile for storage, to be removed immediately before use.

The plunger (16) may be in the form of a dual-seal piston that includes two soft silicone cups that slip over the ends of a double sided Ultem plastic holder, for example. One silicone cup seals on the igniter (13) side and the other silicone cup seals on the drug or injectant (17) side. When the igniter (13) is activated, the double plunger is pushed by the gas pressure, which in turn pushes and pressurizes the injectant (17) which may be liquid. The double plunger is configured to the gas and liquids separate and does not allow leaks.

As mentioned, the nozzle assembly can be inserted into a sleeve, which may be a reusable stainless steel sleeve or a disposable sleeve. The sleeve can provide safety during operation, as a shield covering the nozzle assembly, which may be formed using a plastic. The sleeve can have the same angled taper as the outside of the nozzle assembly, allowing for easy loading and unloading. The sleeve also includes pins that extend outward. The wide end of the sleeve has a notch in the top that accepts the tab in the cap. This aligns the electrical leads with respect to the pins that extend from the side of the sleeve. The sleeve can also have a cylindrical section at the top with a chamfer that allows for easy loading into the plug (11), having a small amount of clearance to the inner diameter of the plug (11). The tip of the nozzle (18) can protrude out the end of the sleeve so that only the sterile portion of the nozzle (18) tip can make contact with the skin.

The nozzle assembly also includes a cap (19), which may be an Ultem cap, configured to hold the igniter into the nozzle assembly. The cap (19) includes holes that allow the igniter leads to protrude therethrough. The holes are aligned to a tab that sticks out the side of the cap (19) for aligning the leads when loading the VISTA-jet.

The plug (11) can be formed using stainless steel and configured to accept a portion of the sleeve. The plug (11) has two slots in the side that guide the sleeve pins and keep them from rotating so the electrical leads in the nozzle assembly are in the correct orientation. The plug (11) has two chamfered holes that allow the electrical leads to stick through. The plug (11) also has alignment pins to guide the integration part (7) so the connectors sticking out of the integration part (7) are correctly oriented to stick in the counter bores of the plug (11). The plug (11) also has a shelf for the lock ring (10) to rest on and threads for the top end slip ring (20) to screw onto, to connect the top end to the bottom end. On the bottom of the plug (11) can include an elastic material to preload the nozzle assembly and sleeve slightly when it is locked into place to keep it there. An O-ring could be used as the elastic material, although other materials, such as a spring or foam, can also be used.

The lock ring (10) can be formed using stainless steel and include channels that guide the sleeve pins when twisted, to form a bayonet style connector. Twisting the lock ring (10) one way will pull up the sleeve and lock it, as well as positioning the nozzle assembly into place. To release the sleeve and nozzle assembly, the lock ring is rotated the opposite way. The channels start straight at the lower end of the lock ring (10) to accept the sleeve and nozzle assembly, and are angled near the top to pull the sleeve up when rotated. The finished position has a small bump that the pins have to slide over and the resting position is stable (so it will not rotate by itself). Exterior surfaces, particularly of the lock ring (10), may be decorated with traction patterns and/or ribs to increase grip.

As described, the nozzle assembly may be configured for single usage. In some embodiments the nozzle assembly may be color coded, bar coded, and/or labeled with large, easy to understand letters (for example, M for morphine). The nozzle assembly and/or sleeve may be configured in a variety of operable designs, depending on application, such as shown in embodiments (1)-(9) of FIG. 3.

The preparation or assembly of a device, as detailed with reference to FIGS. 1-3 is now described. The initiators are one-time use and are sealed in a disposable plastic nozzle assembly with the leads sticking through a cap. The initiators may be sealed in the nozzle assembly with an epoxy (e.g. 3M DP460NS), but may also be sealed during a manufacturing process, such as during an injection molding.

To prep for assembly, the end of the nozzle assembly, bottom side of the cap, and outside diameter of the initiator may be sanded with a rough sandpaper (such as 100 or 200 grit). All parts may be cleaned with isopropyl alcohol and care taken that the nozzle orifice(s) are not clogged. Silicone mold release (Jet Lube) may be applied to the smaller inner bore (0.180″) of the nozzles with a Comfort-In plunger going all the way down to the nozzle end. Silicone mold release is also applied on the outside of the double plunger.

To fill nozzles with the injectant, the 0.180″ bore is slightly overfilled filled with the injectant in the vertical orientation and a double plunger is pushed into the bore using a rod with a slightly smaller bore. The plunger is pushed to a specific depth into the bore leaving a predetermined volume of injectant in the bore. While pushing the plunger down, the rest of the injectant exits the nozzle and no air is allowed in. Once the plunger is in the correct location, a cover is placed over the nozzle end. The current process uses electrical tape and is not sterile. The future manufacturing plan would sterilize everything before this step and fill in a sterile environment. The larger bore chamber and sanded nozzle ends are rewashed with isopropyl alcohol.

To seal in the initiator, the leads are placed through the holes in the cap with the sanded side toward the initiator and at least a 0.2″ gap between the back of the initiator and cap. 3M DP460NS epoxy is applied (at least 1/16″ thick) around the outside diameter of the initiator and at least a 0.2″ diameter amount is applied between the initiator and cap. The initiator with epoxy is loaded into the nozzle body by holding the sides of the cap while twisted the cap and initiator 360 degrees. The epoxy uniformly fills the gap between the initiator outer diameter and nozzle inner diameter and the extra epoxy flows back towards the cap (not into the bore). The end of the initiator seats on the larger bore of the nozzle body and the cap is pushed to the nozzle end with slight pressure (˜1 pound of force) which leaves about a 10-15 mil bond line of the epoxy. The extra epoxy flows out sides between the nozzle and cap and is wiped away. A 1 ml syringe with 25 gauge needle tip is filled with the epoxy the side vent hole of the nozzle is filled. The nozzles are left upright in a glass scintillation vial to cure for a minimum of 48 hours and ideally 72 hours or longer.

The nozzle assemblies are currently made and assembled by hand. The future manufacturing vision is to injection mold the nozzle body and caps in two pieces. The initiator can be injection molded and sealed in the cap end, such as in nylon injection mold. The plunger is added in the other end, sterilized, filled with the drug, and capped in a sterile environment. The two halves are joined with an adhesive or solvent, depending on what material is chosen.

This device is easy to use and can be operated with no gloves, latex gloves, or even thick combat gloves. To load the device, the lock ring is rotated to release the sleeve. The tape is removed from the nozzle end, and the nozzle assembly is loaded into the sleeve aligning the tap sticking out on the cap with the notch in the top of the sleeve. The sleeve and nozzle assembly area loaded into the plug, aligning the sleeve pins with the plug and lock ring notches. The lock ring is rotated so the sleeve pins click into place, which ensures the nozzle assembly and sleeve are properly loaded.

To make an injection, the side button is pushed down and in to arm the device. While holding the side button in that position, the top button is pushed to cause the injection. When making an injection, the nozzle tip is held in contact the skin (or other substrate) with light pressure. The nozzle is preferable held in place on the skin when making an injection, and normal to the skin. To unload, the lock ring is rotated to release the sleeve and nozzle assembly. The nozzle tip may be tapped or pushed to release the used plastic nozzle assembly and discard in the appropriate manner. The device is now ready to be loaded with another nozzle assembly.

A working prototype of the VISTA-jet has been tested and validated, including: injecting on a load cell to measure the force of the micro jet over time, injecting in gel, and injecting in deceased and harvested animal tissue. By injecting on a load cell with nozzle tips that are normal to the surface, the load cell can measure the force from the fluid stream over time at a high data rate. Pressure can be calculated from the force of the stream as well as velocity and jet power. Jet power directly relates to the depth of injections and the jet power can be compared to other COTS NFID's. Validated data included measures of force, pressure, velocity, and jet power over time of a lower pressure nozzle of the VISTA-jet designed to inject to a shallower depth to a higher jet power COTS NFID device.

For testing, 20% polyacrylamide gel (in water) is a consistent and uniform substrate that can be used to characterize different nozzles and injections. It's clear, cheap, and easy to mix and the injection depths and spread can be measured. Typically, an injection from a NFID device injects in a narrow channel then at a certain depth blooms or spreads out in one or more planes. This is likely because the gel is not porous, and once the gel rips, there is a stress concentration at that rip and the fracture can propagate along that plane.

Test injecting was also performed in deceased and harvested animal tissue, such as Wistar rat skin. The skin was previously frozen for storage and is thawed, placed on top of a compliant substrate to simulate muscle tissue, and injected. The VISTA-jet was compared to a COTS device by injecting on the rat skin. The injectant (blue food coloring in water) was measured to encompass a larger area compared to the COTS device.

In summary, the present hand-held injection delivery system provides numerous innovations. These include an energetic source to deliver doses with required speed and penetration, nozzle designs configured to control dosage depth, spread and distribution, a dual-seal piston design configured to prevent contamination between injections, and a housing design that can be integrated with other hand-held devices and/or include functionalities, such as a light, GPS, camera, phone, and so on. The injection delivery system also includes inter-changeable, pre-loaded tips that can be coded by color, shape or other feature to enable safe and reliable selection of injectate and dosage, even in low light conditions. The injection delivery system also includes single shot delivery of one or multiple injectates that are prefilled in separate compartments of the disposable nozzle, where the delivery could be through a single orifice where the drugs or injectants are mixed just before delivery or through multiple orifices in close proximity at the tip of the disposable nozzle.

The injection delivery system provides enhanced safety by a two-button operation that limits accidental injections, such as self-injections. This includes injection activation by pressing a button or sensor that contains an electric switch, such as on the top or side of the VISTA-jet device. The injection delivery system may be configurable to wirelessly identify the unit dose of injectant loaded in the device as well as the patient/provider (armbands/badge). To this end, the system may also include a lock-out feature, where the device is locked, preventing administration of an injectant, if an incorrect correct medication, patient, time of administration, is determined. The injection delivery system may be also be configured to wirelessly interface to enable communication of administered medicines to electronic health records.

The injection delivery system can include disposable nozzle assemblies prefilled with a drug or other injectant, such as medicament, vaccine, nanoparticle sensors, and others. As such, a wide variety of nozzles and drugs can be used in the same VISTA-jet device. In addition, different drugs can be in different nozzles with different volumes and a respective design for different depths of injections, including angled nozzles for shallow large area injections of nanoparticle sensors.

The injection delivery system can include a chemical energetic device that can be used to create pressure to deliver injectant, instead of a large spring or compressed gas. These may be pyrotechnical initiators including a pyrogen, such as a metal oxidizer, e.g. zirconium potassium perchlorate (ZZP), boron-potassium nitrate (BPN or BKNO3), aluminum-potassium perchlorate and titanium-aluminum-potassium perchlorate, or a metal hydride oxidizer, e.g. zirconium hydride—potassium perchlorate (ZHPP) and titanium hydride potassium perchlorate (THPP), intermetallics such as titanium-boron, nickel-aluminum, palladium-aluminum, or others, like cis-bis-(5-nitrotetrazolato) tetraminecobalt(III) perchlorate (BNCP), lead azide, Hexamethylene triperoxide diamine (HMTD), tetrazene explosive, lead mononitro-resorcinates, lead dinitro-resorcinates, and lead trinitro-resorcinates.

The injection delivery system can accommodate varying skin thicknesses, dispense injectant to pre-set delivery depths, dispense injectant to a variety of body locations, accommodate a plurality of medical “payloads,” operate safely in low light and tactile-limiting conditions, as well as for extended periods of time without recharging or replacing power supply.

In some embodiments, a dye or contrast agent may be employed in the injectate to provide a marker for what was injected to hospital/EMT/medic in the field and/or remote location. The marker may be detected with visible, ultraviolet or infrared light depending on specific user needs. In some embodiments, a pressure sensitive adhesive (PSA) can be placed at the end of the nozzle, which may also be color coded and/or bar coded, to assist in later clarifications of what was injected, and/or the PSA bar code affixed onto the patient's chart/skin/jacket and so on, e.g. to improve continuity of care, safety, and improve ease of appropriate billing.

In an embodiment the injection delivery system may be further configured to penetrate a thin membrane over the skin, such as chemical and biological protection suit, so that an injection can be administered without removing the user from the protective suit, preferably without affecting the usability of the suit. This embodiment incorporates a small pinpoint to the tip of the needle free injector to nick the suit and provide a pathway through the suit for the delivery of the jet of medicine. By physically initiating the pathway, the needle free jet of medicine is able to penetrate the suit and the underlying skin of the patient without causing significant openings in the suit.

The pinprick is typically co-located with the injection nozzle aperture so that the injection can pass through the hole provided by the pinprick. The pinprick will not break the skin to any significant depth, but penetrate membrane materials of protective clothing. Thus, the pinprick can serve two purposes—one is to penetrate protective clothing such as chem/bio suits which typically contain a membrane that is difficult to penetrate with standard NFIDs, and the second is to simply retain the NFID nozzle in position (mechanically) to prevent the nozzle from moving out of position during an injection.

The pin prick orifice size generally matches the needle free injector orifice, typically 0.005″ to 0.020″, depending on application. Outside dimensions are selected to be rigid, sturdy and durable enough for field use, to provide the membrane penetration function and to avoid the disadvantages and dangers of hypodermic needles (broken needles, sharps handling procedures, etc.), and depend on the fabrication material; exemplary stainless steel, integrated pin pricks of the device are typically of about 0.5 or 1 mm to about 3 or 5 mm long and wide.

In one aspect the invention provides a multi-use needle-free injection device comprising connectable proximate and distal sections, wherein the proximate section comprises a power supply, actuator, circuitry and a housing, and the distal section comprises a disposable nozzle tip prefilled with an injectate, a chemical energetic device, and a dual seal piston barrier between the energetic device and the injectate, wherein the proximate and distal sections and their components operably linked and configured for single-hand injecting of the injectate.

Embodiments include:
wherein the disposable nozzle tip is circumscribed with a metal sleeve or shroud, and a bayonet connector comprising pins protruding from the sleeve or shroud aligns and locks onto the proximate section, such as by rotating a lock ring with angled channels or by snap-locking an elastic retaining clip.
wherein the actuator is a button activated electric switch.
wherein the proximate section further comprises a second safety switch configured to reduce or eliminate unintended injections/misfires
wherein the proximate section further comprises connector components configured to make electric and mechanical connection to the distal section.
wherein the distal section comprises a disposable cartridge containing the nozzle, energetic device and piston barrier.
wherein the nozzle tip comprises a non-hypodermic pin prick configured to penetrate a protective clothing barrier membrane but not underlying skin, such as wherein the pin prick is integrated, 0.25-2 or 0.5-1.5, 0.75-1.25, 0.9-1.1 or about 1 mm long, stainless steel, and/or has an orifice diameter 0.005″ to 0.020″.

The invention also provides a kit comprising a subject device and a set of disposable nozzle tips prefilled with injectate.

The invention also provides a method of using a subject device comprising pressing the distal end against skin and activating the actuator, wherein the actuator signals the power supply through the circuitry to trigger the energetic device to create a pressure gradient which translocates the piston barrier and ejects the injectate out an orifice of the nozzle into a microjet that penetrates the skin.

The invention encompasses all combinations of recited particular and preferred embodiments. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

What is claimed is:

1. A needle-free injection delivery system comprising:
a distal section having a disposable nozzle assembly that comprises:
a chemical igniter sealed inside a nozzle body,
an injectate stored inside a nozzle tip, and connected to the igniter, and
a plunger, forming a sealed barrier between the injectate and the chemical igniter, the plunger configured to introduce the injectate into a subject upon activation of the chemical igniter; and
a proximal section, removably coupled to the distal section, and comprising a housing having therein electrical circuitry configured to control the activation of the chemical igniter.

2. The system of claim 1, wherein the chemical igniter is a chemical energetic device.

3. The system of claim 1, wherein the chemical igniter comprises an energetic material selected from a list comprising zirconium potassium perchlorate (ZZP), boron-potassium nitrate (BPN or BKNO3), aluminum-potassium perchlorate and titanium-aluminum-potassium perchlorate, zirconium hydride—potassium perchlorate (ZHPP), titanium hydride potassium perchlorate (THPP), titanium-boron, nickel-aluminum, palladium-aluminum, cis-bis-(5-nitrotetrazolato)tetraminecobalt(III) perchlorate (BNCP), lead azide, Hexamethylene triperoxide diamine (HMTD), tetrazene explosive, lead mononitro-resorcinates, lead dinitro-resorcinates, and lead trinitro-resorcinates.

4. The system of claim 1, wherein the nozzle assembly further comprises an expansion chamber between the chemical igniter and the plunger, the expansion chamber being configured to control a depth of penetration of the injectant inside the subject.

5. The system of claim 1, wherein at least one of the chemical igniter and expansion chamber is configured to generate a pressure in a range between 3000 and 6000 psi.

6. The system of claim 1, wherein the nozzle assembly further comprises a sleeve that circumscribes at least a portion of the nozzle body.

7. The system of claim 1, wherein the nozzle assembly is coupled to an integration part attached to the proximal section using a plug and lock ring.

8. The system of claim 1, wherein the circuitry further comprises an actuator operated by a user that is configured to activate the chemical igniter to generate a pressure gradient inside the nozzle assembly sufficient to translocate the plunger inside a bore and introduce the injectate into the subject.

9. The system of claim 1, wherein the circuitry further comprises a charging switch operated by a user and configured to allow or prevent the activation of the chemical igniter.

10. The system of claim 1, wherein the nozzle tip further comprises at least one opening through which the injectate traverses, the at least one opening configured to generate a microjet that pierces a skin of the subject.

11. The system of claim 10, wherein the at least one opening has a diameter in a range between 0.005″ to 0.020″.

12. The system of claim 10, wherein the at least one opening extends radially outward.

13. The system of claim 10, wherein the injectant comprises at least one of a drug, a medicament, a vaccine, a dye, a contrast agent, and nanoparticle sensors.

14. The system of claim 1, wherein the nozzle tip further comprises a non-hypodermic pin prick configured to penetrate a membrane covering the skin of the subject.

15. The system of claim 14, wherein the non-hypodermic pin prick has an orifice diameter in a range between 0.005″ to 0.020″, and a length and width in a range between 0.25 mm to 3 mm.

16. The system of claim 1, wherein proximate section further comprises connector components configured to make an electrical connection and a mechanical connection to the distal section.

17. The system of claim 1, wherein the system further comprises a kit including a set of disposable nozzle tips prefilled with the injectate.

18. A method for operating a needle-free injection delivery system comprising a chemical igniter housed inside a nozzle body of a disposable nozzle assembly, an injectate connected to the chemical igniter and stored inside a nozzle tip of the disposable nozzle assembly, and a plunger forming a seal barrier between the chemical igniter and injectate, the method comprising:
pressing the nozzle tip against a skin of a subject;
operating a charging switch to allow activation of the chemical igniter; and
operating an actuator to activate the chemical igniter.

19. The method of claim 18, wherein the chemical igniter is configured to generate a pressure gradient inside the disposable nozzle assembly sufficient to translocate the plunger and introduce the injectate into the skin of the subject.

20. The method of claim 19, wherein the pressure gradient includes pressures in a range between 3000 and 6000 psi.
